SEQUENCE LISTING (17 sequences)

SEQ ID NO: 1 - protein, 480 aa, Artificial Sequence
  Description of Artificial Sequence Synthetic polypeptide
VPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIG

SEQ ID NO: 2 - protein, 480 aa, Artificial Sequence
  Description of Artificial Sequence Synthetic polypeptide
VPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSG

SEQ ID NO: 3 - protein, 480 aa, Artificial Sequence
  Description of Artificial Sequence Synthetic polypeptide
VPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIG

SEQ ID NO: 4 - protein, 5 aa, Artificial Sequence
  Description of Artificial Sequence Synthetic peptide
  MOD_RES (4)..(4): Any amino acid
  See specification as filed for detailed description of substitutions and preferred embodiments
VPGXG

SEQ ID NO: 5 - protein, 5 aa, Artificial Sequence
  Description of Artificial Sequence Synthetic peptide
  See specification as filed for detailed description of substitutions and preferred embodiments
VPGSG

SEQ ID NO: 6 - protein, 5 aa, Artificial Sequence
  Description of Artificial Sequence Synthetic peptide
  See specification as filed for detailed description of substitutions and preferred embodiments
VPGVG

SEQ ID NO: 7 - protein, 7 aa, Artificial Sequence
  Description of Artificial Sequence Synthetic peptide
GLVPRGS

SEQ ID NO: 8 - protein, 119 aa, Homo sapiens
EDASSDSTGADPAQEAGTSKPNEEISGPAEPASPPETTTTAQETSAAAVQGTAKVTSSRQELNPLKSIVEKSILLTEQALAKAGKGMHGGVPGGKQFIENGSEFAQKLLKKFSLLKPWA

SEQ ID NO: 9 - protein, 140 aa, Artificial Sequence
  Description of Artificial Sequence Synthetic polypeptide
  MOD_RES (133)..(133): Any amino acid
  MOD_RES (138)..(138): Any amino acid
  See specification as filed for detailed description of substitutions and preferred embodiments
MGEDASSDSTGADPAQEAGTSKPNEEISGPAEPASPPETTTTAQETSAAAVQGTAKVTSSRQELNPLKSIVEKSILLTEQALAKAGKGMHGGVPGGKQFIENGSEFAQKLLKKFSLLKPWAGLVPRGSGVPGXGVPGXGY

SEQ ID NO: 10 - protein, 138 aa, Homo sapiens
MKFTTLLFLAAVAGALVYAEDASSDSTGADPAQEAGTSKPNEEISGPAEPASPPETTTTAQETSAAAVQGTAKVTSSRQELNPLKSIVEKSILLTEQALAKAGKGMHGGVPGGKQFIENGSEFAQKLLKKFSLLKPWA

SEQ ID NO: 11 - DNA, 406 bp, Artificial Sequence
  Description of Artificial Sequence Synthetic polynucleotide
catatggaagacgcttcttctgactctaccggtgctgacccggctcaggaagctggtacctctaaaccgaacgaagaaatctctggtccggctgaaccggcttctccgccggaaaccaccaccaccgctcaggaaacctctgctgctgctgttcagggtaccgctaaagttacctcttctcgtcaggaactgaacccgctgaaatctatcgttgaaaaatctatcctgctgaccgaacaggctctggctaaagctggtaaaggtatgcacggtggtgttccgggtggtaaacagttcatcgaaaacggttctgaattcgctcagaaactgctgaaaaaattctctctgctgaaaccgtgggctggtctggttccgcgtggttctggttactgatctcctcggatcc

SEQ ID NO: 12 - protein, 7 aa, Artificial Sequence
  Description of Artificial Sequence Synthetic peptide
GLVPRGS

SEQ ID NO: 13 - protein, 12 aa, Artificial Sequence
  Description of Artificial Sequence Synthetic peptide
GVPGSGVPGIGY

SEQ ID NO: 14 - protein, 482 aa, Artificial Sequence
  Description of Artificial Sequence Synthetic polypeptide
GVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGY

SEQ ID NO: 15 - protein, 240 aa, Artificial Sequence
  Description of Artificial Sequence Synthetic polypeptide
VPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSG

SEQ ID NO: 16 - protein, 240 aa, Artificial Sequence
  Description of Artificial Sequence Synthetic polypeptide
VPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIG

SEQ ID NO: 17 - protein, 8 aa, Artificial Sequence
  Description of Artificial Sequence Synthetic peptide
GLVPRGSG

(19) United States
(12) Patent Application Publication
(10) Pub. No.: US 2018/0094137 A1
(43) Pub. Date: Apr. 5, 2018

(54) COMPOSITIONS AND METHODS FOR THE DELIVERY OF DRUGS TO THE OCULAR SURFACE BY CONTACT LENSES

(71) Applicant: University of Virginia Patent Foundation, Charlottesville, VA (US)

(72) Inventors: John Andrew Mackay, Los Angeles, CA (US); Wan Wang, Los Angeles, CA (US); Gordon W. Laurie, Charlottesville, VA (US)

(73) Assignee: University of virginia Patent Foundation, Charlottesville, VA (US)

(21) Appl. No.: 14/781,226

(22) PCT Filed: Mar. 31, 2014
(86) PCT No.: PCT/US14/32412

§ 371 Date: Sep. 29, 2015

Related U.S. Application Data
(60) Provisional application No. 61/806,558, filed on Mar. 29, 2013.

(51) Int. Cl. C08L 89/00; C07K 14/78; C07K 14/475; A61K 47/64; A61K 47/65; A61K 9/00; G02C 7/04; G02B 1/04

(52) U.S. Cl. CPC C08L 89/00; C07K 14/78; C07K 14/475; A61K 47/6435; C07K 2319/00; A61K 9/0051; G02C 7/049; G02B 1/043; C08L 2203/02; A61K 47/65

(57) ABSTRACT

Disclosed herein are novel methods and compositions for targeting ocular diseases. One aspect relates to a contact lens comprising an elastin-like peptide (ELP) component and optionally a therapeutic agent. Also provided are methods for treating ocular diseases comprising administering a contact lens of the disclosure to a subject in need thereof.

This application claims the benefit under 35 U.S.C. § 119(e) of U.S. Provisional Application Ser. No. 61/806,558, filed Mar. 29, 2013, the contents of which is hereby incorporated by reference into the present disclosure.

### BACKGROUND

Accounting for approximately 90% of all ophthalmic medications, topical ophthalmic solutions (eye drops) have long been the most commonly used method of ocular drug delivery. However, eye drops are generally considered an inefficient drug delivery system that is characterized by a transient overdose, followed by a relatively short period of effective therapeutic concentration, and then a prolonged period of insufficient concentration or underdosing.

Ophthalmic ointments, an alternative to liquid eye drops, have a longer contact time with the cornea and possibly provide higher chance for drug absorption than a solution due to their high viscosity. Nevertheless as each drop is diluted, the majority of the active agent is washed away by reflex tearing, blinking, or drained through the nasolacrimal system so that only 1 to 7% of an eye drop is absorbed by the eye. To remedy this problem, collagen shields have been proposed to absorb and then slowly release a wide variety of medications. In one application, these shields are applied after surgical procedures involving the corneal epithelium promote re-epithelialization and delivery antibiotic prophylaxis. However, collagen shields are not widely used for daily drug delivery because they lack optical clarity, are difficult to self-insert, are uncomfortable to the patient, and degrade quickly.

Beyond providing millions of people with glasses-free vision correction, contact lenses have been proposed as a more comfortable way to therapeutically manage ocular anterior segment disorders. Indications for using soft contact lenses therapeutically include protecting a compromised ocular surface, pain management, and promoting epithelialization or wound closure. Many studies report that contact lenses improve the corneal penetration and bioavailability of topically applied pharmaceutical agents using two approaches. In the first method, lenses are soaked in the drug solution for a period of time and then placed on the eye, resulting in a high initial release, followed by a slower, long-term release during hours to days of lens wearing. This method is commonly employed with antibiotics and non-steroidal anti-inflammatory drugs (NSAIDs) postoperatively, and with antibiotics for severe infections. Alternatively, a topical drug can be applied over the lens while the lens is in situ. This approach is necessary when a patient wears a contact lens as a protective device (bandage lens) following a corneal injury or a serious infection, in which case a lens is used as a shield or bandage lens to promote wound repair. The lens absorbs drug from the tear film and then acts as a reservoir, slowly releasing the drug into the tears as the overall concentration of the drug in the tear film declines. Both these approaches prolong the contact time of the drug with the cornea and thus improve penetration of drugs into the cornea. Ongoing research of drug-eluting contact lenses includes copolymerizing the contact lens' hydrogel material (p-HEMA) with other polymers, such as PLGA; releasing drug from microemulsions contained in hydrogel prototype lenses; molecularly imprinted hydrogels, and immobilizing drug-containing liposomes onto the surface of contact lenses. However, achieving sustained, long-term drug delivery at the normal physiological temperature, pH, and salinity of human eye still remains a challenge. Furthermore, it would be desirable to develop contact lens drug carriers that are relatively simple in design: which do not require complicated and expensive manufacturing processes; which do not impair or interfere with the patient's vision; and which do not require a substantial change in the practice patterns of ophthalmologists.

### SUMMARY

To develop new treatments and delivery mechanisms for ocular diseases, new drug vehicles are required that are biocompatible, biodegradable and easily modified with bioactive peptides. An emerging approach to this challenge employs protein polymers to drive reversible assembly of nanostructures. As one example, the elastin-like-polypeptides (ELPs) possess unique phase transition behavior, which mediates self-assembly of nanoparticles. As they are composed from amino acids, protein polymers and ELPs may be produced either by chemical synthesis or by biological expression from an engineered gene expressed by a host cell.

This document discloses the useful interaction between biocompatible polymeric used for contact lenses and therapeutic materials composed from protein polymers, such as ELPs, that adhere to promote long-term delivery of peptide therapeutics for the enhanced treatment of ocular diseases and disorders.

Thus, in one aspect, this disclosure describes a biocompatible, polymeric material that comprises, or alternatively consists essentially of, or yet further consists of a biocompatible, polymeric material and an ELP. In one aspect, the polymeric material is a material comprising one or more of poly(hydroxyl ethyl methacrylate), methacrylic acid, N-vinyl pyrrolidone, cyclohexyl methacrylate, N,N-dimethyl acrylamide or a contact lens material, non-limiting examples of such are provide in Table 1 although any suitable biocompatible, polymeric material may be used and therefore, the disclosure is not so limited. In one aspect the ELP is attached to the polymeric material in a random fashion or alternatively, attached in a pre-determined design such one or more concentric rings, or only in the center of the polymeric material or alternatively, only around the periphery of the polymeric material, or alternatively in swirls or stamped blocked or rectangular geometries. In one particular aspect, the ELP is attached to the polymer in one or more concentric rings.

The ELP component of the polymeric material can be any ELP known in the art which includes those obtained from either chemical or biological synthetic routes. In one aspect, the ELP is a diblock polypeptide. Non-limiting examples of such include one or more of the ELPs described herein, which optionally includes one or more of SEQ ID NOS: 1 to 6, or a biological equivalent of each thereof. In a further aspect, the polymeric material-ELP is combined with a pharmaceutically acceptable carrier, such as saline or the like, to maintain the polymeric material's biocompatible characteristics.

In a further aspect, the polymeric material further comprises, or consists essentially of, or yet further consists of a detectable label, e.g., a fluorophore or a detectable dye.

In one aspect, the polymeric material and ELP further comprises a therapeutic agent bound to the ELP or encapsulated within the ELP. Non-limiting examples of a therapeutic agent include a peptide, a protein, an antibody, an antibody fragment or a small molecule. In a different aspect, the therapeutic acts as a growth factor, an anti-microbial agent or a non-steroidal anti-inflammatory drug. In a further aspect, an effective amount of the therapeutic is bound to the ELP and polymeric material. In different further aspects, the polymeric material-ELP is combined with a pharmaceutically acceptable carrier, such as saline or the like, to maintain the therapeutic agent's effectiveness and/or the polymeric material's biocompatible characteristics.

Methods to link or encapsulate a therapeutic agent to the ELP are described in International Application No.: PCT/US2013/64719, filed Oct. 11, 2013, incorporated by reference herein. In one embodiment, the therapeutic agent is fused to the ELP by covalent attachment to a cleavable peptide sequence located between the therapeutic agent and the ELP In one aspect, the cleavable peptide sequence is a thrombin cleavable peptide sequence which comprises the amino acid sequence GLVPR₁GS (SEQ ID NO.: 7), or a biological equivalent thereof, wherein a biological equivalent is a sequence having at least 80% sequence identity, or alternatively at least 90% sequence identity, or alternatively at least 95% sequence identity to SEQ ID NO: 7, or a sequence that hybridizes under conditions of high stringency to the polynucleotide that encodes the sequence or its complement.

In one aspect, the therapeutic agent is the lacritin protein, a fragment of lacritin, or a biological equivalent thereof. This strategy is used to delivery other peptide therapeutics including growth factors, including but not limited to Epidermal Growth Factor (EGF), transforming growth factor beta (TGF-β), human growth factor (HGF). In one embodiment, the lacritin comprises SEQ ID NO.: 8 or 10, or a biological equivalent of each thereof, which is covalently fused to the ELP either directly or via a cleavable peptide sequence located between the therapeutic agent and the ELP. To biologically express a fusion peptide between an ELP and a therapeutic, DNA encoding a cleavable peptide sequence is inserted between that encoding for the therapeutic agent and ELP sequence. Using conventional molecular biology methodology, the resulting gene fusion can be cloned into a plasmid that encodes for antibiotic resistance and transformed into prokaryotic or eukaryotic host cells, including but not limited to BLR(DE3) competent cells, Origami™ B competent cells, or HEK-293 cells. Transformants expressing the fusion protein can be isolated using antibiotic selection, such as ampicillin, kanamycin, or gentamycin. Alternatively, the cleavage sequence can be added between ELPs and the therapeutic peptide that are produced using solid-phase peptide synthesis. Alternatively, cleavage sequence can be added after/before therapeutic agent protein sequence using solid-phase peptide synthesis. In one aspect, the cleavable peptide sequence is a thrombin cleavable peptide sequence which comprises the amino acid sequence GLVPRIGS (SEQ ID NO. 7), or a biological equivalent thereof (as defined herein). A non-limiting example of the lacritin-ELP amino acid sequence comprises the amino acid sequence of SEQ ID NO: 9 or a biological equivalent thereof.

This disclosure also provide the isolated polynucleotides that encode the polypeptide ELPs as described above which can be contained within an expression vector for recombinant expression in a host cell. Accordingly, the isolated host cells comprising the isolated polynucleotides and/or ELP polypeptides, that in one aspect contain the therapeutic peptide, are within the scope of this disclosure. Methods to prepare the ELP alone or in combination with the therapeutic are within the scope of this disclosure and described herein. Methods to prepare the polymeric material of this disclosure is further provided, as is the method comprising absorbing, conjugating, or coating a polymeric material with the ELP, alone or in combination with the therapeutic agent.

The polymeric materials as described herein are useful for delivering a therapeutic agent, the method comprising contacting the polymeric material topically or internally, with a subject to be treated. As used herein, the term “subject” intends an animal, such as a mammal, e.g., a canine, an equine, a rabbit, or feline, or a human patient. In one aspect, the polymeric material is in contact with the ocular surface of an eye. Thus, the polymeric material is useful in methods for treating an ocular disease, comprising contacting the polymeric material with the eye of a patient in need of such treatment. Non-limiting examples of an ocular disease that can be treated by this method includes without limitation dry eye, age-related macular degeneration, diabetic retinopathy, retinal venous occlusions, retinal arterial occlusion, macular edema, postoperative inflammation, infection, dryness, uveitis retinitis, proliferative vitreoretinopathy and glaucoma.

This disclosure also provides a kit comprising one or more polymeric materials, alone or in combination with a therapeutic agent and instructions for use as described herein.

### BRIEF DESCRIPTION OF THE FIGURES

FIGS. 1A-D show NHS-Rhodamine labeled V96 (Rho-V96) selectively phase separated onto Proclear Contact lens. A) Rho-V96 on SDS-PAGE. B) Reversible temperature dependent phase transition behavior of Rho-V96 at 500 μM. C) Fluorescence image of contact lenses after Rho-V96 phase separation. D) Contact lens modification process at 37° C. *1. Proclear Compatibles contact lens (CooperVision); 2. Dailies (CIBA Vision); 3. Acuvue OASYS (Johnson & Johnson Vision Care); 4. Acuvue Advance Plus (Johnson & Johnson Vision Care). Pictures were captured using Bio-Rad VersaDoc MP System.

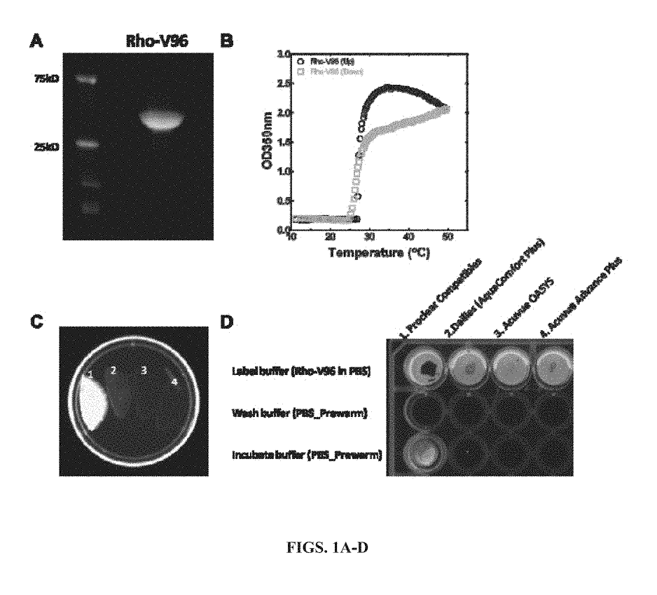

FIGS. 2A-D show that the protein polymer architecture controls rate of release from contact lenses. A) A therapeutic protein, lacritin, fused to an ELP. B) ELPs adhere and release from contact lenses. C) Representative rhodamine-ELP labeled Proclear contact lenses. D) Confocal laser scanning microscopy of rhodamine-labeled ELPs incubated with contact lenses at 37° C. Soluble ELPs (Rho-S96) washed away immediately. ELP nanoparticles (Rho-S48I48) form embedded puncta within the lens. ELP coacervate (Rho-V96) uniformly stains the lens, which was retained at high levels after 3 days. Scale bar: 50 μm.

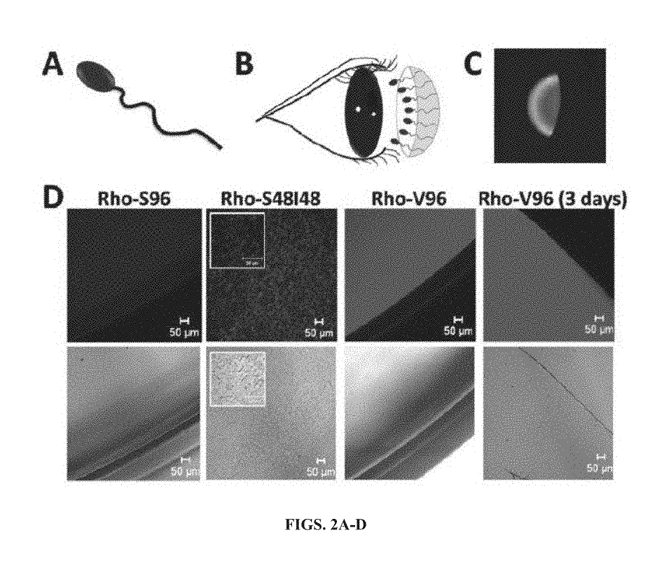

FIGS. 3A-E show the ELP phase transition extended retention time of Rho-V96 on contact lens. A) Proclear contact lenses were incubated with equal concentration of Rho-V96 (100 μM) at 4° C. and 37° C. for overnight. 37° C. incubation showed significantly higher ELP retention than 4° C. B) Proclear contact lens incubated in Rho-V96 (100 μM) at 37° C. was cut into two halves and incubated at 37° C. or 4° C. ddH2O for overnight. Contact lens incubated at 37° C. exhibited higher Rho-V96 retention. C, D&E) Rho-V96 phase separation onto Proclear contact lens is a reversible process. Three individual lenses were modified with Rho-V96 at 37° C., gently washed in PBS at 37° C. and incubated in 2 ml PBS for one week (168 h). Equal amount of sample (100 μl) was taken out from incubating solution at each time point (0 h, 30 min, 1 h, 2 h, 4 h, 8 h, 12 h, 24 h, 48 h, 96 h, 168 h). After one week, lenses were incubated in 1.5 ml fresh PBS at 4° C. for 24 h (Wash 1) with gentle shaking. Washing step was repeated once in another 1.5 ml fresh PBS at 4° C. for 24 h (Wash 2) with gentle shaking. After measuring fluorescence of all collected samples using fluorescent plate reader (D), samples were concentrated to equal volume using Amicon Ultra protein concentrator (3 kD cut-off). Equal volume of samples was loaded onto 4-20% gradient gel and imaged using Bio-Rad VersaDoc MP System (C). Protein retention ratio was characterized using ImageJ (E). 96.2±1.8% of total fluorescence and 99.1±2.3% of total protein was retained on the lens after one week incubation at 37° C.

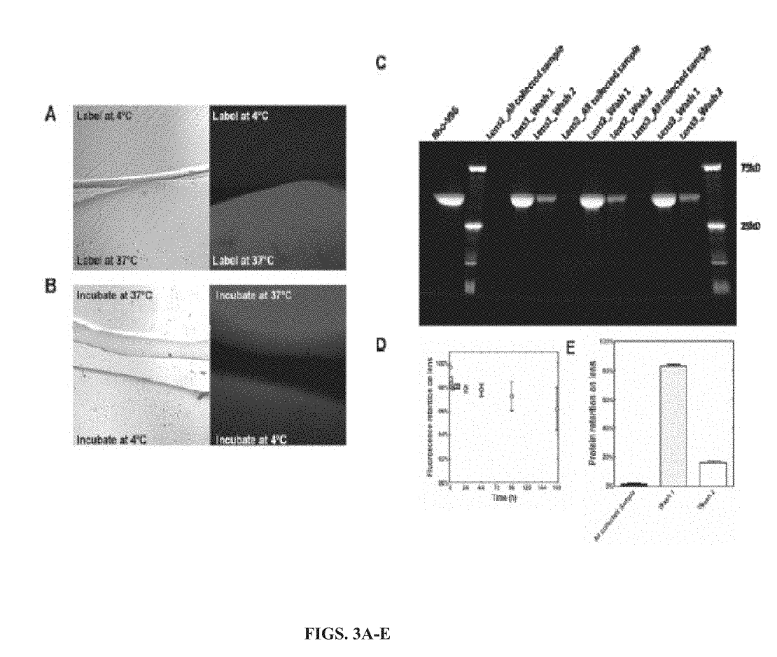

FIGS. 4A and B show that ELP selectively phase separate onto Proclear Compatibles™ contact lens. A) Among four types of contact lens tested, rhodamine labeled V96 selectively phase separated onto Proclear Compatibles™ contact lens. 1: Proclear Compatibles™; 2: Dailies (AquaComfort Plus); 3: Acuvue OASYS; 4: Acuvue Advance Plus. B) Different shapes of rhodamine labeled V96 on Proclear Compatibles™ contact lens. Upper: white light; lower: fluorescence.

FIGS. 5 A to E show Tₜ and temperature dependent affinity of ELPs towards Proclear Compatible™ contact lens. A) Representative picture showing different affinity of V96 and S96 to the lens at 37° C. and 4° C. after 24 h incubation. B) Total fluorescence intensity quantification result showing ELPs' attachment to the lens was Tₜ and temperature dependent. C) Group one exhibited high retention on the lens after one week incubation at 37° C. D) Group two, three and four showed similar release pattern and can fit into same two-phase decay model. E) Group five illustrated different release kinetics from group one, with significant lower plateau. ***p<0.001; grey line: predicted values using one phase decay model; grey dash line: predicted values using two phase decay model.

FIGS. 6A to G show spatiotemporal HCE-T cell uptake. A) Representative pictures showing time dependent uptake of Lac and Lac-V96 into HCE-T cells. B-C Quantification result showing V96 tag modulated cell uptake speed and amount of exogenous Lac. D) Cartoon showing rho-Lac-V96 “ring” modified contact lens with three representative regions. 1: rho-Lac-V96 fully covered cell region; 2: cell region half covered by rho-Lac-V96; 3: cell region not covered by the lens. E-G) Representative pictures showing HCE-T cell uptake of rho-Lac-V96 in three regions delivered by contact lens. Red: rhodamine; Blue: DAPI staining of nuclei.

### DETAILED DESCRIPTION

### Definitions

The practice of the present invention will employ, unless otherwise indicated, conventional techniques of tissue culture, immunology, molecular biology, microbiology, cell biology and recombinant DNA, which are within the skill of the art. See, e.g., Sambrook et al., (1989) Molecular Cloning: A Laboratory Manual, 2nd edition; Ausubel et al., eds. (1987) Current Protocols In Molecular Biology; MacPherson, B. D. Hames and G. R. Taylor eds., (1995) PCR 2: A Practical Approach; Harlow and Lane, eds. (1988) Antibodies, A Laboratory Manual; Harlow and Lane, eds. (1999) Using Antibodies, a Laboratory Manual; and R. I. Freshney, ed. (1987) Animal Cell Culture.

All numerical designations, e.g., pH, temperature, time, concentration, and molecular weight, including ranges, are approximations which are varied (+) or (−) by increments of 1.0 or 0.1, as appropriate. It is to be understood, although not always explicitly stated that all numerical designations are preceded by the term “about”. It also is to be understood, although not always explicitly stated, that the reagents described herein are merely exemplary and that equivalents of such are known in the art.

As used in the specification and claims, the singular form “a,” “an” and “the” include plural references unless the context clearly dictates otherwise.

As used herein, the term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of” shall mean excluding more than trace elements of other ingredients and substantial method steps. Embodiments defined by each of these transition terms are within the scope of this invention.

The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

The term “therapeutic agent” refers to an agent or component capable of inducing a biological effect in vivo and/or in vitro, such as for example an anti-inflammation agent, an antibiotic, a polypeptides and diverse protein/antibody therapeutic libraries via encapsulation or recombinant protein expression, a small molecule, a nucleic acid, a protein, antibody, antibody fragment or a polypeptide. Strategies for incorporation of therapeutic agents are described in International Patent Appl. No: PCT/US2013/64719, filed Oct. 11, 2013. Non-limiting examples of therapeutic agents include lactrintin, cyclosporin A, ketorolac, nepafenac, bromfenac; antibiotics such as Bacitracin, Erythromycin; growth factors such as Epidermal Growth Factor (EGF), Transforming Growth Factor Beta (TGF-β), Hepatocyte Growth Factor (HGF); protease inhibitors such as Matrix Metallopeptidase 2 (MMP-2) or Matrix Metallopeptidase 9 (MMP-9). The biological effect may be useful for treating and/or preventing a condition, disorder, or disease in a subject or patient.

As used herein, the term “biological equivalent thereof” is used synonymously with “equivalent” unless otherwise specifically intended. When referring to a reference protein, polypeptide or nucleic acid, the term intends those having minimal homology while still maintaining desired structure or functionality. Unless specifically recited herein, it is contemplated that any polynucleotide, polypeptide or protein mentioned herein also includes equivalents thereof. For example, an equivalent intends at least about 60%, or 65%, or 70%, or 75%, or 80% homology or identity and alternatively, at least about 85%, or alternatively at least about 90%, or alternatively at least about 95%, or alternatively 98% percent homology or identity and exhibits substantially equivalent biological activity to the reference protein, polypeptide or nucleic acid. Alternatively, a biological equivalent is a peptide encoded by a nucleic acid that hybridizes under stringent conditions to a nucleic acid or complement that encodes the peptide. Hybridization reactions can be performed under conditions of different “stringency”. In general, a low stringency hybridization reaction is carried out at about 40° C. in about 10×SSC or a solution of equivalent ionic strength/temperature. A moderate stringency hybridization is typically performed at about 50° C. in about 6×SSC, and a high stringency hybridization reaction is generally performed at about 60° C. in about 1×SSC. Hybridization reactions can also be performed under “physiological conditions” which is well known to one of skill in the art. A non-limiting example of a physiological condition is the temperature, ionic strength, pH and concentration of Mg²⁺ normally found in a cell.

A polynucleotide or polynucleotide region (or a polypeptide or polypeptide region) having a certain percentage (for example, about 60%, 65%, 70%, 75%, 80%, 85%, 90%, 95% or 97%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

An “equivalent” is used in the alternative with “biological equivalent” of a polynucleotide or polypeptide refers to a polynucleotide or a polypeptide having a substantial homology or identity to the reference polynucleotide or polypeptide. In one aspect, a “substantial homology” is greater than about 60%, 65%, 70%, 75%, 80%, 85%, 90%, 95% or 98% homology.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in an eukaryotic cell.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

“Regulatory polynucleotide sequences” intends any one or more of promoters, operons, enhancers, as known to those skilled in the art to facilitate and enhance expression of polynucleotides.

An “expression vehicle” is a vehicle or a vector, non-limiting examples of which include viral vectors or plasmids, that assist with or facilitate expression of a gene or polynucleotide that has been inserted into the vehicle or vector.

A “delivery vehicle” is a vehicle or a vector that assists with the delivery of an exogenous polynucleotide into a target cell. The delivery vehicle may assist with expression or it may not, such as traditional calcium phosphate transfection compositions.

A “composition” is intended to mean a combination of active agent and another compound or composition, inert (for example, the polymeric material of this disclosure, a solid support or pharmaceutically acceptable carrier) or active, such as an adjuvant.

A “pharmaceutical composition” is intended to include the combination of an active agent with a carrier, inert or active (e.g., the polymeric material of this disclosure), making the composition suitable for diagnostic or therapeutic use in vitro, in vivo or ex vivo.

“An effective amount” refers to the amount of an active agent or a pharmaceutical composition sufficient to induce a desired biological and/or therapeutic result. That result can be alleviation of the signs, symptoms, or causes of a disease, or any other desired alteration of a biological system. The effective amount will vary depending upon the health condition or disease stage of the subject being treated, timing of administration, the manner of administration and the like, all of which can be determined readily by one of ordinary skill in the art.

As used herein, the terms “treating,” “treatment” and the like are used herein to mean obtaining a desired pharmacologic and/or physiologic effect. The effect may be prophylactic in terms of completely or partially preventing a disorder or sign or symptom thereof, and/or may be therapeutic in terms of a partial or complete cure for a disorder and/or adverse effect attributable to the disorder.

As used herein, to “treat” further includes systemic amelioration of the symptoms associated with the pathology and/or a delay in onset of symptoms. Clinical and sub-clinical evidence of “treatment” will vary with the pathology, the subject and the treatment.

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art.

The agents and compositions of the present invention can be used in the manufacture of medicaments and for the treatment of humans and other animals by administration in accordance with conventional procedures, such as an active ingredient in pharmaceutical compositions.

As used herein, the term “detectable label” intends a directly or indirectly detectable compound or composition that is conjugated directly or indirectly to the composition to be detected, e.g., N-terminal histidine tags (N-His), magnetically active isotopes, e.g., ¹¹⁵Sn, ¹¹⁷Sn and ¹¹⁹Sn, a non-radioactive isotopes such as ¹³C and ¹⁵N, polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to magnetically active isotopes, non-radioactive isotopes, radioisotopes, fluorochromes, luminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescent labeling assay components are known in the art and described for example in Haugland, Richard P. (1996) Handbook of Fluorescent Probes and Research Chemicals (6^{th} ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6^{th} ed.).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

The term “contact lens” refers to the entire product that is placed in contact with the cornea. Non-limiting examples of commercially available contact lenses are listed in Table 1. Generally, contact lenses comprise a central lens with a diameter of 7-9 mm. The central lens of conventional contact lenses contains front and back surface curvatures that combine to create the optical power of the lens (after accounting for lens thickness and refractive index of the lens material). In addition to the central lens, contact lenses also conventionally comprise an outer region. The outer region is located between the edge of the central lens and the edge of the contact lens. Conventionally, the outer region of a contact lens is designed to provide a comfortable fitting of the lens to the eye, and causes minimal physiological disruption to the normal functioning of the eye. The contact lenses utilized in the present disclosure may be customized or conventional contact lenses designed based on the unique low and high order aberrations of each individual eye. Conventional contact lenses correct low order aberrations (myopia, hyperopia and astigmatism), whereas customized contact lenses also correct high order aberrations including optical characteristics such as coma, spherical aberration and trefoil. Preferably, the forces applied to the eye by the upper and/or lower eyelid are dispersed so that the optical characteristics of the eye are unchanged by downward gaze or near work. Contact lenses used in the present disclosure may also be corrective (i.e. corrects a vision defect) or non-corrective (i.e. no correction of vision defect). In certain embodiments, the contact lens may be non-corrective and serve as a drug carrier for the efficient delivery of therapeutic agents and proteins to the eye of a patient in need thereof. U.S. patent publication 2011/0230588 describes methods of making hydrogel or soft contact lenses. Table 1 shows a summary of contact lenses commercially available. Both traditional and p-HEMA hydrogel contact lenses and silicone hydrogel contact lenses are included within this disclosure.

TABLE 1
Contact lens material characteristics

| | | Listed | Measured | | |
|---|---|---|---|---|---|
| | | water | water | Oxygen | |
| Commercial | | content | volume^{a} | permeability | FDA |
| Name (supplier) | Polymer composition | (%) | (mL) | (barrers^{b}) | category |
| Optima FW | Polymacon p- | 38 | 0.01048 | 9 | Group I |
| (Bausch & Lomb, | HEMA/NVP/CMA | | | | (non-ionic, |
| Rochester, NY) | | | | | low water |
| | | | | | content) |
| Focus Night & | Lotrafilcon A | 24 | 0.00448 | 140 | Group I |
| Day (Ciba Vision, | DMA/Siloxane macromer | | | | (non-ionic, |
| Duluth, GA) | | | | | low water |
| | | | | | content) |
| Softlens 66 | Alphafilcon A p- | 66 | 0.0231 | 30 | Group II |
| (Bausch & Lomb, | HEMA/NVP/CMA | | | | (non-ionic, |
| Rochester, NY) | | | | | high water |
| | | | | | content) |
| Proclear | Omafilcon A p- | 59 | 0.01741 | 22 | Group II |
| Compatibles | HEMA/phosphorylcholine | | | | (non-ionic, |
| (Biocompatibles, | | | | | high water |
| Norfolk, VA) | | | | | content) |
| PureVision | Balafilcon A Siloxane | 36 | 0.00956 | 99 | Group III |
| (Bausch & Lomb, | macromer/NVP | | | | (ionic, low |
| Rochester, NY) | | | | | water |
| | | | | | content) |
| Acuvue/Surevue | Etafilcon A p-HEMA/MA | 58 | 0.01588 | 21 | Group IV |
| (Johnson & | | | | | (ionic, high |
| Johnson, | | | | | water |
| Jackonsonville, | | | | | content) |
| FL) | | | | | |
| Focus Monthly | Vifilcon A p- | 55 | 0.01588 | 19 | Group IV |
| (Ciba Vision, | HEMA/MA/NVP | | | | (ionic, high |
| Duluth, GA) | | | | | water |
| | | | | | content) |

p-HEMA, poly(hydroxyl ethyl methacrylate);
MA, methacrylic acid;
NVP, N-vinyl pyrrolidone;
CMA, cyclohexyl methacrylate;
DMA, N,N-dimethyl acrylamide.
^{a}Volume of water in the contact lens as obtained by subtracting the wet lens from the dry lens weight.
^{b}1 barrer =10⁻¹¹ (cm³mL O₂)/s mL mmHg), obtained from the contact lens package inserts.

By “biocompatible,” it is meant that the components of the delivery system will not cause tissue injury or injury to the human biological system. To impart biocompatibility, polymers and excipients that have had history of safe use in humans or with GRAS (Generally Accepted As Safe) status, will be used preferentially. For a composition to be biocompatible, and be regarded as non-toxic, it must not cause toxicity to cells.

The term “polymeric material”, refers a material comprising a polymer matrix and a plurality of interconnecting pores. Non-limiting examples of polymeric materials include polymers comprising unsaturated carboxylic acids, such as methacrylic acid and acrylic acid; (meth)acrylic substituted alcohols, such as 2-hydroxyethylmethacrylate and 2-hydroxyethylacrylate; vinyl lactams, such as N-vinyl pyrrolidone; (meth)acrylamides, such as methacrylamide and N,N-dimethylacrylamide, poly(hydroxyl ethyl methacrylate); N-vinyl pyrrolidone; and cyclohexyl methacrylate.

The term “fuse,” “fused” or “link” refers to the covalent linkage between two polypeptides in a fusion protein. The polypeptides are typically joined via a peptide bond, either directly to each other or via an amino acid linker. Optionally, the peptides can be joined via non-peptide covalent linkages known to those of skill in the art.

The term “cleavable peptide” refers to a peptide that may be cleaved by a molecule or protein. By way of example, cleavable peptide spacers include, without limitation, a peptide sequence recognized by proteases (in vitro or in vivo) of varying type, such as Tev, thrombin, factor Xa, plasmin (blood proteases), metalloproteases, cathepsins (e.g., GFLG, etc.), and proteases found in other corporeal compartments. In some embodiments employing cleavable peptides, the fusion protein (i.e. therapeutic protein) may be inactive, less active, or less potent as a fusion, which is then activated upon cleavage of the spacer in vivo.

As used herein, the term “elastin-like peptide (ELP) component” intends a polypeptide that forms stable nanoparticle (also known as a micelle) above the transition temperature of the ELP. In another aspect, the ELP component comprises, or alternatively consists essentially of, or yet further consists of the polypeptide S48I48 having the sequence G(VPGSG)n(VPGIG)nY (SEQ ID NO: 1) or a biological equivalent thereof, wherein n is an integer that denotes the number of repeats, and can be from about 6 to about 192, or alternatively from about 15 to 75, or alternatively from about 40 to 60, or alternatively from about 45 to 55, or alternatively about 48), wherein in one aspect, S48I48 comprises, or alternatively consists essentially of, or yet further consists of the amino acid sequence G(VPGSG)₄₈(VPGIG)₄₈Y, or a biological equivalent thereof. A biological equivalent of polypeptide S48I48 is a peptide that has at least 80% sequence identity to polypeptide S48I48 or a peptide encoded by a polynucleotide that hybridizes under conditions of high stringency to a polynucleotide that encodes polypeptide S48I48 or its complement, wherein conditions of high stringency comprise hybridization reaction at about 60° C. in about 1×SSC. The biological equivalent will retain the characteristic or function of forming a nanoparticle (also known as a micelle) when the biological equivalent is raised above the transition temperature of the biological equivalent or, for example, the transition temperature of S48I48.

In another aspect, the ELP comprises, or alternatively consists essentially of, or yet further consists of, the polypeptide (VPGSG)n (SEQ ID NO: 5) or a biological equivalent thereof, wherein n is an integer that denotes the number of repeats, and can be from about 6 to about 192, or alternatively from about 15 to 125, or alternatively from about 50 to 110, or alternatively from about 90 to 100, or alternatively about 96 (VPGSG)₉₆ (SEQ ID NO: 2) or a biological equivalent of each thereof. A biological equivalent of polypeptide is a peptide that has at least 80% sequence identity to SEQ ID No: 2 or a peptide encoded by a polynucleotide that hybridizes under conditions of high stringency to a polynucleotide that encodes SEQ ID NOS: 2 or 5, or the respective complements, wherein conditions of high stringency comprise hybridization reaction at about 60° C. in about 1×SSC. The biological equivalent will retain the characteristic or function of forming a nanoparticle (also known as a micelle) when the biological equivalent is raised above the transition temperature of the biological equivalent or, for example, the transition temperature of SEQ ID NOS: 2 or 5.

In another aspect, the ELP comprises, or alternatively consists essentially of, or yet further consists of, the polypeptide (VPGVG)n (SEQ ID NO: 6) or a biological equivalent thereof, wherein n is an integer that denotes the number of repeats, and can be from about 6 to about 192, or alternatively from about 15 to 125, or alternatively from about 50 to 110, or alternatively from about 90 to 100, or alternatively about 96) (VPGIG)₉₆ (SEQ ID NO: 3) or a biological equivalent of each thereof. A biological equivalent of polypeptide is a peptide that has at least 80% sequence identity to SEQ ID NOS: 3 or 6, or a peptide encoded by a polynucleotide that hybridizes under conditions of high stringency to a polynucleotide that encodes SEQ ID NOS: 3 or 6, or its complement, wherein conditions of high stringency comprise hybridization reaction at about 60° C. in about 1×SSC. The biological equivalent will retain the characteristic or function of forming a nanoparticle (also known as a micelle) when the biological equivalent is raised above the transition temperature of the biological equivalent or, for example, the transition temperature of SEQ ID NOS: 3 or 6.

In another aspect, the ELP comprises, or alternatively consists essentially of, or yet further consists of, the repeated pentapeptide sequences, (VPGXG)ₙ (SEQ ID NO: 4) or a biological equivalent thereof, derived from human tropoelastin, where X is the “guest residue” which is any amino acid and n is the number of repeats. In one aspect, n is an integer from about 6 to about 192, or alternatively from about 15 to 75, or alternatively from about 40 to 60, or alternatively from about 45 to 55, or alternatively about 48). A biological equivalent of polypeptide (VPGXG)ₙ is a peptide that has at least 80% sequence identity to polypeptide, or a peptide encoded by a polynucleotide that hybridizes under conditions of high stringency to a polynucleotide that encodes polypeptide (VPGXG)ₙ or its complement, wherein conditions of high stringency comprise hybridization reaction at about 60° C. in about 1×SSC. The biological equivalent will retain the characteristic or function of forming a nanoparticle (also known as a micelle) when the biological equivalent is raised above the transition temperature of the biological equivalent or, for example, the transition temperature of (VPGXG)ₙ.

### Elastin-Like Polypeptides (ELPs)

This disclosure relates to polymeric materials and contact lenses comprising genetically engineered polypeptide nanoparticles. To develop new treatments for ocular diseases, new drug carriers are required that are biocompatible and easily modified with bioactive peptides. An emerging solution to this challenge utilizes genetically engineered polypeptides to drive the assembly of nanostructures.

Elastin-like-polypeptides (ELPs) are a genetically engineered polypeptide with unique phase behavior (see for e.g. S. R. MacEwan, et al., Biopolymers 94(1) (2010) 60-77) which promotes recombinant expression, protein purification and self-assembly of nanostructures (see for e.g. A. Chilkoti, et al., Advanced Drug Delivery Reviews 54 (2002) 1093-1111). In one aspect, the ELP is as described above, e.g., any one or more of SEQ ID NOS: 1 to 6 or a biological equivalent thereof.

In another aspect the ELP is composed of repeated pentapeptide sequences, (VPGXG)ₙ (SEQ ID. NO: 4) derived from human tropoelastin, where X is the “guest residue” which is any amino acid and n is the number of repeats or a biological equivalent thereof. In one aspect, n is an integer from about 6 to about 192, or alternatively from about 15 to 75, or alternatively from about 40 to 60, or alternatively from about 45 to 55, or alternatively about 48). A biological equivalent of polypeptide (VPGXG)ₙ is a peptide that has at least 80% sequence identity to polypeptide, or a peptide encoded by a polynucleotide that hybridizes under conditions of high stringency to a polynucleotide that encodes polypeptide (VPGXG)ₙ or its complement, wherein conditions of high stringency comprise hybridization reaction at about 60° C. in about 1×SSC. The biological equivalent will retain the characteristic or function of forming a nanoparticle (also known as a micelle) when the biological equivalent is raised above the transition temperature of the biological equivalent or, for example, the transition temperature of (VPGXG)ₙ. In one embodiment, X is any amino acid except proline. This peptide motif displays rapid and reversible de-mixing from aqueous solutions above a transition temperature, Tₜ. Below Tₜ, ELPs adopt a highly water soluble random coil conformation; however, above Tₜ, they separate from solution, coalescing into a second aqueous phase. The Tₜ of ELPs can be tuned by choosing the guest residue and ELP chain length as well as fusion peptides at the design level (see for e.g. MacEwan S R, et al., Biopolymers 94(1): 60-77). The ELP phase is both biocompatible and highly specific for ELPs or ELP fusion proteins, even in complex biological mixtures. Genetically engineered ELPs are monodisperse, biodegradable, non-toxic. Throughout this description, ELPs are identified by the single letter amino acid code of the guest residue followed by the number of repeat units, n. For example, S48I48 represents a diblock copolymer ELP with 48 serine (S) pentamers ([VPGSG]₄₈, SEQ ID. NO: 5) at the amino terminus and 48 isoleucine (I) pentamers ([VPGIG]₄₈, SEQ ID. NO: 6) at the carboxy terminus. A “diblock” as used herein refers to an ELP with two blocks of repeated polypeptide sequence. For example, the diblock (VPGSG) 48 (VPGIG) 48 (SEQ ID. NO: 1) comprises 48 repeated units of a polypeptide having the sequence VPGSG (SEQ ID NO: 5) and 48 repeated units of a polypeptide having the sequence VPGIG (SEQ ID. NO: 6). In one embodiment, the ELP component comprises a polypeptide with the sequence of SEQ ID. NO: 1. In each of the above embodiments, the ELP can comprise a biological equivalent thereof.

In further embodiments, the ELP component comprises, consists essentially of, or yet consists of, a polypeptide with the sequence (VPGSG)₄₈(VPGIG)₄₈ (SEQ ID. NO: 1), (VPGSG)₉₆ (SEQ ID. NO: 2) or (VPGVG)₉₆ (SEQ ID. NO: 3) or a biological equivalent or each thereof.

Described herein are ELP fusion proteins, which can be self-assembled into nanoparticles. The diameter of the nanoparticle can be from about 1 to about 1000 nm or from about 1 to about 500 nm, or from about 1 to about 100 nm, or from about 1 to about 50 nm, or from about 20 to about 50 nm, or from about 30 to about 50 nm, or from about 35 to about 45 nm. In one embodiment, the diameter is about 40 nm.

In one embodiment, the ELP component may further comprise a therapeutic agent. ELP fusion proteins are able to conjugate small molecules, such as, for example, chemotherapeutic agents, anti-inflammation agents, antibiotics and polypeptides and other water soluble drugs. In addition, the ELP nanoparticles are useful for carrying DNA, RNA, protein and peptide-based therapeutics.

ELPs have potential advantages over chemically synthesized polymers as drug delivery agents. First, because they are biosynthesized from a genetically encoded template, ELPs can be made with precise molecular weight. Chemical synthesis of long linear polymers does not typically produce an exact length, but instead a range of lengths. Consequently, fractions containing both small and large polymers yield mixed pharmacokinetics and biodistribution. Second, ELP biosynthesis produces very complex amino acid sequences with nearly perfect reproducibility. This enables very precise selection of the location of drug attachment. The drug can be selectively placed on the corona, buried in the core, or dispersed equally throughout the polymer. Third, ELP can self-assemble into multivalent nanoparticles that can have excellent site-specific accumulation and drug carrying properties. Fourth, because ELP are designed from native amino acid sequences found extensively in the human body they are biodegradable, biocompatible, and tolerated by the immune system. Fifth, ELPs undergo an inverse phase transition temperature, Tₜ, above which they phase separate into large aggregates. By localized heating, additional ELP can be drawn into the target site, which may be beneficial for increasing drug concentrations.

A therapeutic agent such as a drug, for example, may be attached to the ELP through cysteine, lysine, glutamic acid or aspartic acid residues present in the polymer. In some embodiments, the cysteine, lysine, glutamic acid or aspartic acid residues are generally present throughout the length of the polymer. In some embodiments, the cysteine, lysine, glutamic acid or aspartic acid residues are clustered at the end of the polymer. In some embodiments of the presently described subject matter, therapeutics are attached to the cysteine residues of the ELP using thiol reactive linkers. In some embodiments of the presently described subject matter, therapeutics are attached to the lysine residues of the high molecular weight polymer sequence using NHS (N-hydroxysuccinimide) chemistry to modify the primary amine group present on these residues. In some embodiments of the presently described subject matter, therapeutics are attached to the glutamic acid or aspartic acid residues of the ELP using EDC (1-Ethyl-3-[3-dimethylaminopropyl]carbodiimide Hydrochloride) chemistry to modify the carboxylic acid group present on the ELP residues.

The therapeutic associated with the ELP may be hydrophobic or hydrophilic. For hydrophobic drugs, attachment to the terminus of the ELP may facilitate formation of the multivalent nanoparticle. The number of drug particles attached to the ELP can be from about 1 to about 30, or from about 1 to about 10, or about 1, 2, 3, 4, 5, 6, 7, 8, 9, or 10. In some embodiments, the attachment points for a therapeutic are equally distributed along the backbone of the ELP, and the resulting drug-ELP is prevented from forming nanoparticle structures under physiological salt and temperature conditions.

In certain embodiments, the therapeutic agent is an anti-microbial agent or a non-steroidal anti-inflammatory drug. As used herein, the term “anti-microbial” is meant to include prevention, inhibition, termination, or reduction of virulence factor expression or function of a microbe. “Prevention” can be considered, for example, to be the obstruction or hindrance of any potential microbial growth. “Termination” can be considered, for example, to be actual killing of the microbes by the presence of the composition. “Inhibition” can be considered, for example, to be a reduction in microbial growth or inhibiting virulence factor expression or function of the microbe. As used herein, the term “anti-microbial agent” is meant to encompass any molecule, chemical entity, composition, drug, therapeutic agent, or biological agent capable of preventing or reducing growth of a microbe, or capable of blocking the ability of a microbe to cause disease. An example of an anti-microbial agent is an antibiotic. The term includes small molecule compounds, antisense reagents, siRNA reagents, antibodies, enzymes, peptides, organic or inorganic molecules, natural or synthetic compounds and the like.

In a further embodiment, the therapeutic agent is a non-steroidal anti-inflammatory drug. The term “non-steroidal anti-inflammatory drug,” usually abbreviated to NSAIDs, but also referred to as nonsteroidal anti-inflammatory agents/analgesics (NSAIAs) or nonsteroidal anti-inflammatory medicines (NSAIMs), are a class of drugs that provide analgesic and antipyretic (fever-reducing) effects, and, in higher doses, anti-inflammatory effects. The term “nonsteroidal” distinguishes these drugs from steroids, which, among a broad range of other effects, have a similar eicosanoid-depressing, anti-inflammatory action. As analgesics, NSAIDs are unusual in that they are non-narcotic. Non-limiting examples of NSAIDs include aspirin, ibuprofen, and naproxen.

The methods, lenses, and compositions of the present invention are effective against bacteria including, for example, gram-positive and gram-negative cocci, gram positive and gram negative straight, curved and helical/vibroid and branched rods, sheathed bacteria, sulfur-oxidizing bacteria, sulfur or sulfate-reducing bacteria, spirochetes, actinomycetes and related genera, myxobacteria, mycoplasmas, rickettsias and chlamydias, cyanobacteria, archea, fungi, parasites, viruses and algae. In particular, the present invention is useful against the Pseudomonas species of bacteria, e.g., Pseudomonas aeruginosa, and other microbes that are found in the eye.

In addition to therapeutics, the ELPs may also be associated with a detectable label that allows for the visual detection of in vivo uptake of the ELPs. Suitable labels include, for example, fluorescein, rhodamine, tetramethylrhodamine, eosin, erythrosin, coumarin, methyl-coumarins, pyrene, Malacite green, Alexa-Fluor®, stilbene, Lucifer Yellow, Cascade Blue™, and Texas Red. Other suitable optical dyes are described in Haugland, Richard P. (1996) Molecular Probes Handbook.

In certain embodiments, the ELP components include polymeric or oligomeric repeats of the pentapeptide VPGXG (SEQ ID. NO: 4), where the guest residue X is any amino acid, that in one aspect, excludes proline. X may be a naturally occurring or non-naturally occurring amino acid. In some embodiments, X is selected from alanine, arginine, asparagine, aspartic acid, cysteine, glutamic acid, glutamine, glycine, histidine, isoleucine, leucine, lysine, methionine, phenylalanine, serine, threonine, tryptophan, tyrosine and valine. In some embodiments, X is a natural amino acid other than proline or cysteine.

The guest residue X may be a non-classical (non-genetically encoded) amino acid. Examples of non-classical amino acids include: D-isomers of the common amino acids, 2, 4-diaminobutyric acid, α-amino isobutyric acid, A-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, C α-methyl amino acids, N α-methyl amino acids, and amino acid analogs in general.

Selection of X is independent in each ELP structural unit (e.g., for each structural unit defined herein having a guest residue X). For example, X may be independently selected for each structural unit as an amino acid having a positively charged side chain, an amino acid having a negatively charged side chain, or an amino acid having a neutral side chain, including in some embodiments, a hydrophobic side chain.

In each embodiment, the structural units, or in some cases polymeric or oligomeric repeats, of the ELP sequences may be separated by one or more amino acid residues that do not eliminate the overall effect of the molecule, that is, in imparting certain improvements to the therapeutic component as described. In certain embodiments, such one or more amino acids also do not eliminate or substantially affect the phase transition properties of the ELP component (relative to the deletion of such one or more amino acids).

The ELP component in some embodiments is selected or designed to provide a Tₜ ranging from about 10 to about 80° C., such as from about 10 to about 60° C., or from about 38 to about 45° C. The transition temperature, in some embodiments, is above the body temperature of the subject or patient (e.g., >37° C.) thereby remaining soluble in vivo, or in other embodiments, the Tₜ is below the body temperature (e.g., <37° C.) to provide alternative advantages, such as in vivo formation of a drug depot for sustained release of the therapeutic agent.

The Tₜ of the ELP component can be modified by varying ELP chain length, as the Tₜ generally increases with decreasing MW. For polypeptides having a molecular weight>100,000, the hydrophobicity scale developed by Urry et al. (PCT/US96/05186, which is hereby incorporated by reference in its entirety) is preferred for predicting the approximate Tₜ of a specific ELP sequence. However, in some embodiments, ELP component length can be kept relatively small, while maintaining a target Tₜ, by incorporating a larger fraction of hydrophobic guest residues (e.g., amino acid residues having hydrophobic side chains) in the ELP sequence. For polypeptides having a molecular weight<100,000, the Tₜ may be predicted or determined by the following quadratic function: Tₜ=M₀+M₁X+M₂X² where X is the MW of the fusion protein, and M₀=116.21; M₁=−1.7499; M₂=0.010349.

While the Tₜ of the ELP component, and therefore of the ELP component coupled to a therapeutic component, is affected by the identity and hydrophobicity of the guest residue, X, additional properties of the molecule may also be affected. Such properties include, but are not limited to solubility, bioavailability, persistence, and half-life of the molecule.

### Therapeutic Peptides

In certain embodiments of the disclosure, the ELP component further comprises a therapeutic protein. The term “therapeutic protein” as used herein is a protein that may be used to treat a disease, particularly an ocular disease. Non-limiting examples of therapeutic proteins include lacritin, anti-VEGF proteins or antibodies or therapeutics, such as afibercept (Eylea®), bevacizumab (Avasin®), pegaptanib (Macugen®) or ranibuzumab (Lucentis®); Rab Escort Protein-1(REP-1, described in Ophthalmic Genetic. (2012) June; 33(2):57-65); Retinitis Pigmentosa Related 65 (described in Hum Gene Ther. Clin Dev. (2013) March: 24(1):23-8); ATP-binding cassette transporter abc4 (ABC4, described in. (Nat Genet. 1997 March; 15(3):236-46); and MYO7A (associated with The Usher Syndromes (“USH”) (Ophthalmology. 2014 February; 121(2):580-7.). The therapeutic protein may also be an antibody that provides therapy for a condition of the eye. When therapeutic proteins and/or therapeutic agents are added to the polymeric material or contact lens of the disclosure, they are typically added in an effective amount or a therapeutically effective amount.

In one embodiment, the therapeutic protein is lacritin or a biological equivalent thereof. Lacritin is a glycoprotein encoded in humans by the LACRT gene. Lacritin is a secreted protein found in tears and saliva. Lacritin also promotes tear secretion and proliferation of some epithelial cells. Lacritin is thus a prosecretory mitogen. Functional studies suggest a role in epithelial renewal of some nongermative epithelia. By flowing downstream through ducts, it may generate a ‘proliferative field’. Lacritin also promotes secretion. This raises the possibility that lacritin may have clinical applications in the treatment of dry eye, the most common eye disease. The lacritin protein sequence is known in the art. For example, the GenBank Accession Nos.: NP_150593 and AAG44392.1 represents the lacritin sequence. The sequence associated with this GenBank Accesion number is herein incorporated by reference in its entirety.

In one embodiment, lacritin comprises the amino acid sequence of SEQ ID NO: 8 or a biological equivalent thereof:

(SEQ ID. NO: 8)
EDASSDSTGADPAQEAGTSKPNEEISGPAEPASPPETTTTAQETSAAAVQ
GTAKVTSSRQELNPLKSIVEKSILLTEQALAKAGKGMHGGVPGGKQFIEN
GSEFAQKLLKKFSLLKPWA.

A biological equivalent is as described above.

In a further embodiment, the lacritin ELP comprises the amino acid sequence of SEQ ID NO: 9 or a biological equivalent thereof as described above: MGEDASSDSTGADPAQEAGTSKPNEEISGPAEPASPPETTTTAQETSAAAVQGTAKVTSS RQELNPLKSIVEKSILLTEQALAKAGKGMHGGVPGGKQFIENGSEFAQKLLKKFSLLKP WAGLVPR|GSG(VPGX₁G)ₙ₁(VPGX₂G)ₙ₂Y (SEQ ID NO: 9); wherein X₁ and X₂ represent a guest residue as defined herein and n1 and n2 represents the repeat number of pentamers (VPGXG) (SEQ ID NO: 4) as described above.

In a further embodiment, lacritin comprises the amino acid sequence of SEQ ID NO: 10 or a biological equivalent thereof:

(SEQ ID NO: 10)
MKFTTLLFLAAVAGALVYAEDASSDSTGADPAQEAGTSKPNEEISGPAEP
ASPPETTTTAQETSAAAVQGTAKVTSSRQELNPLKSIVEKSILLTEQALA
KAGKGMHGGVPGGKQFIENGSEFAQKLLKKFSLLKPWA.

The following polynucleotide sequence represents an example of a lacritin polynucleotide sequence:

(SEQ ID NO: 11)
5′-CATATGGAAGACGCTTCTTCTGACTCTACCGGTGCTGACCCGGCTCA
GGAAGCTGGTACCTCTAAACCGAACGAAGAAATCTCTGGTCCGGCTGAAC
CGGCTTCTCCGCCGGAAACCACCACCACCGCTCAGGAAACCTCTGCTGCT
GCTGTTCAGGGTACCGCTAAAGTTACCTCTTCTCGTCAGGAACTGAACCC
GCTGAAATCTATCGTTGAAAAATCTATCCTGCTGACCGAACAGGCTCTGG
CTAAAGCTGGTAAAGGTATGCACGGTGGTGTTCCGGGTGGTAAACAGTTC
ATCGAAAACGGTTCTGAATTCGCTCAGAAACTGCTGAAAAAATTCTCTCT
GCTGAAACCGTGGGCTGGTCTGGTTCCGCGTGGTTCTGGTTACTGATCTC
CTCGGATCC-3′.

The term “biological equivalent” is defined above. In one aspect, a biological equivalent is a peptide encoded by a nucleic acid that hybrizes to a nucleic acid that encodes the lacritin protein or its complement under conditions of a high stringency hybridization reaction, that is performed at about 60° C. in about 1×SSC that has substantial identical biological activity to the above-noted sequence.

When a therapeutic protein is part of the ELP component, the therapeutic protein may be fused to the N- or C-terminus of the ELP component. In one embodiment, a cleavable peptide sequence is between the ELP component and the therapeutic peptide or therapeutic agent. In a related embodiment, the cleavable peptide sequence is a protease cleavage site. The term “protease cleavage site” refers to a peptide sequence that is cleaved by a protease. A protease is any enzyme that conducts proteolysis. Protease cleavage proteins and their cleavage sites are known in the art. Non-limiting examples of proteases include, but are not limited to, for example, thrombin, intracellular proteases, including caspases; proteases involved in the regulation of complement activation; proteases involved in the regulation of coagulation; proteases involved in the regulation of signal transduction; and, proteases involved in the expression or activity of prostaglandins (e.g., PGHS-2). Other proteases include, but are not limited to, matrix metalloproteinases, elastase, alphas-proteinase, proteinase 3, chymotrypsin, trypsin, human mast cell chymase, stratum corneum chymotryptic enzyme, human cathepsin G, bovine chymotrypsin, pig chymotrypsin, tryptase, human leukocyte elastase, pig pancreatic elastase, stratum corneum chymotryptic enzyme. In one embodiment, the protease is thrombin, and the protease cleavage site is a thrombin cleavage site as known in the art and as described herein. In another embodiment, the protease is a protease endogenous to the eye or secretions of the eye (i.e. tears). Proteases endogenous to the eye or eye secretions include, for example, metalloproteinases (MMPs) such as MMP-2 and MMP-9, trypsin-like protease, multicatalytic endopeptidase complex, membrane bound proteases, and calpain.

To insert the cleavable sequence, the cDNA of cleavable peptide sequences is inserted between the cDNA encoding the therapeutic agent and cDNA encoding ELP sequence. The whole therapeutic-cleavage-ELP cDNA is cloned into a chosen plasmid (such as pIDTSmart, Ampicillin resistance) and expressed in either prokaryotic and/or eukaryotic cells, such as BLR(DE3) competent cells, Origami™ B competent cells and HEK-293 cell line, etc. Alternatively, cleavage sequence can be added after/before therapeutic agent protein sequence using solid-phase peptide synthesis. In one aspect, the cleavable peptide sequence is a thrombin cleavable peptide sequence which comprises the amino acid sequence GLVPR|GS (SEQ ID NO.: 12), or a biological equivalent thereof (as defined herein).

### Expression of Recombinant Proteins

ELPs and other recombinant proteins described herein can be prepared by expressing polynucleotides encoding the polypeptide sequences of this invention in an appropriate host cell, i.e., a prokaryotic or eukaryotic host cell. This can be accomplished by methods of recombinant DNA technology known to those skilled in the art. It is known to those skilled in the art that modifications can be made to any peptide to provide it with altered properties. Polypeptides of the invention can be modified to include unnatural amino acids. Thus, the peptides may comprise D-amino acids, a combination of D- and L-amino acids, and various “designer” amino acids (e.g., β-methyl amino acids, C-α-methyl amino acids, and N-α-methyl amino acids, etc.) to convey special properties to peptides. Additionally, by assigning specific amino acids at specific coupling steps, peptides with α-helices, β turns, β sheets, α-turns, and cyclic peptides can be generated. Generally, it is believed that beta-turn spiral secondary structure or random secondary structure is preferred.

The ELPs can be expressed and purified from a suitable host cell system. Suitable host cells include prokaryotic and eukaryotic cells, which include, but are not limited to bacterial cells, yeast cells, insect cells, animal cells, mammalian cells, murine cells, rat cells, sheep cells, simian cells and human cells. Examples of bacterial cells include Escherichia coli, Salmonella enterica and Streptococcus gordonii. In one embodiment, the host cell is E. coli. The cells can be purchased from a commercial vendor such as the American Type Culture Collection (ATCC, Rockville Md., USA) or cultured from an isolate using methods known in the art. Examples of suitable eukaryotic cells include, but are not limited to 293T HEK cells, as well as the hamster cell line BHK-21; the murine cell lines designated NIH3T3, NS0, C127, the simian cell lines COS, Vero; and the human cell lines HeLa, PER.C6 (commercially available from Crucell) U-937 and Hep G2. A non-limiting example of insect cells include Spodoptera frugiperda. Examples of yeast useful for expression include, but are not limited to Saccharomyces, Schizosaccharomyces, Hansenula, Candida, Torulopsis, Yarrowia, or Pichia. See e.g., U.S. Pat. Nos. 4,812,405; 4,818,700; 4,929,555; 5,736,383; 5,955,349; 5,888,768 and 6,258,559.

### Protein Purification

The phase transition behavior of the ELPs allows for easy purification. The ELPs may also be purified from host cells using methods known to those skilled in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide or polypeptide are filtration, ion-exchange chromatography, exclusion chromatography, polyacrylamide gel electrophoresis, affinity chromatography, or isoelectric focusing. A particularly efficient method of purifying peptides is fast protein liquid chromatography or even HPLC. In the case of ELP compositions protein purification may also be aided by the thermal transition properties of the ELP domain as described in U.S. Pat. No. 6,852,834.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

### Pharmaceutical Compositions

Pharmaceutical compositions are further provided. The compositions comprise a carrier and ELPs as described herein. The carriers can be one or more of a solid support or a pharmaceutically acceptable carrier. In one aspect, the compositions are formulated with one or more pharmaceutically acceptable excipients, diluents, carriers and/or adjuvants. In addition, embodiments of the compositions include ELPs, formulated with one or more pharmaceutically acceptable auxiliary substances.

The invention provides pharmaceutical formulations in which the one or more of an isolated polypeptide of the invention, an isolated polynucleotide of the invention, a vector of the invention, an isolated host cell of the invention, or an antibody of the invention can be formulated into preparations for injection in accordance with the invention by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives or other antimicrobial agents. A non-limiting example of such is a antimicrobial agent such as other vaccine components such as surface antigens, e.g. a Type IV Pilin protein (see Jurcisek and Bakaletz (2007) J. of Bacteriology 189(10):3868-3875) and antibacterial agents.

Aerosol formulations provided by the invention can be administered via inhalation. For example, embodiments of the pharmaceutical formulations of the invention comprise a compound of the invention formulated into pressurized acceptable propellants such as dichlorodifluoromethane, propane, nitrogen and the like.

Embodiments of the pharmaceutical formulations of the invention include those in which the ELP is formulated in an injectable composition. Injectable pharmaceutical formulations of the invention are prepared as liquid solutions or suspensions; or as solid forms suitable for solution in, or suspension in, liquid vehicles prior to injection. The preparation may also be emulsified or the active ingredient encapsulated in liposome vehicles in accordance with other embodiments of the pharmaceutical formulations of the invention.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Methods of preparing such dosage forms are known, or will be apparent upon consideration of this disclosure, to those skilled in the art. See, e.g., Remington's Pharmaceutical Sciences, Mack Publishing Company, Easton, Pa., 17th edition, 1985. The composition or formulation to be administered will, in any event, contain a quantity of the compound adequate to achieve the desired state in the subject being treated.

Routes of administration applicable to the ELP compositions described herein include intranasal, intramuscular, subcutaneous, intradermal, topical application, intravenous, nasal, oral, inhalation, intralacrimal, retrolacrimal profusal along the duct, intralacrimal, and other enteral and parenteral routes of administration. Routes of administration may be combined, if desired, or adjusted depending upon the agent and/or the desired effect. An active agent can be administered in a single dose or in multiple doses. Embodiments of these methods and routes suitable for delivery, include systemic or localized routes. In one embodiment, the composition comprising the ELP is administered intralacrimally through injection. In further embodiments, the composition is administered systemically, topically on top of the eye, by retrolacrimal profusion, or intranasally. In embodiments of the contact lenses as described herein, the route of administration is through the eye.

### Treatment of Disease

Methods and compositions disclosed herein are useful in treating disorders and diseases of the eye (ocular diseases) and may be particularly useful to encapsulate or attach drugs for treating disorders localized to the eye.

Certain aspects relate to a method for treating an ocular disease, comprising administering to a patient in need of such treatment a polymeric material or contact lens as described herein. The term “ocular disease” refers to any disorder of the eye and/or lacrimal system. It includes non-infectious ocular diseases such as non-infectious ocular surface diseases, e.g., dry eye, and infectious ocular disease such as those ocular diseases caused by microbes. Diseases treatable by the methods of the present invention include, but are not limited to, diseases of the eyelid such as infectious and non-infectious blepharitis, hordeolum, preseptal cellulites, chalazion, herpes zoster ophthalmicus, dacryocystitis, herpes simplex blepharitis, orbital cellulites, and entropion; diseases of the conjunctiva and sclera, such as allergic conjunctivitis, vernal keratoconjunctivitis, viral conjunctivitis, bacterial conjunctivitis, episcleritis, scleritis, pingueculitis, ocular cysticercosis, toxic follicular conjunctivitis, and giant papillary conjunctivitis; diseases of the cornea, such as keratitis sicca or dry eye syndrome, herpes simplex keratitis, bacterial keratitis, sterile corneal infiltrates, and Salzmann's nodular degeneration; diseases of the uvea, such as inflammatory glaucoma and uveitis; and diseases of the vitreous and retina. Also treated by the contact lenses and methods described herein are contact lens associated diseases or conditions are treatable by the methods of the present invention, e.g., bacterial keratitis, contact lens associated red eye (“CLARE”), contact lens induced peripheral ulcers (“CLPU”) and infiltrative keratitis (“IK”). Further examples of ocular disorders treated by the contact lenses and methods disclosed herein are age-related macular degeneration, Sjögren's syndrome, autoimmune exocrinopathy, diabetic retinopathy, graft versus host disease (exocrinopathy associated with) retinal venous occlusions, retinal arterial occlusion, macular edema, postoperative inflammation, uveitis retinitis, proliferative vitreoretinopathy and glaucoma. In one embodiment, the disease is Sjögren's syndrome. In another embodiment, the disease is keratoconjunctivitis sicca (dry eye). In another embodiment the disease is scleritis. In another embodiment the disease is glaucoma.

Also provided is a method for delivering a drug comprising an elastin-like peptide (ELP) component to the eye, comprising contacting the eye with a contact lens as described herein. In one embodiment, the contact lens is in contact with the ocular surface of the eye.

### Combination Treatments

Administration of the therapeutic agent or substance of the present invention to a patient will follow general protocols for the administration of that particular secondary therapy, taking into account the toxicity, if any, of the treatment. It is expected that the treatment cycles would be repeated as necessary. It also is contemplated that various standard therapies, as well as surgical intervention, may be applied in combination with the described therapy and/or use of the polymeric material

The following examples are intended to illustrate and not limit the invention.

### Example 1

Decorating contact lenses with ELPs will provide a platform for hundreds of potential therapeutic entities (including small molecules, peptides, proteins and monoclonal antibodies) to function in a new format: a bioadhesive drug reservoir. For example, the bio-construction of lacritin-ELP library with various hydrophobicity and nanoparticle sizes ranging from 2-3 nm (lacritin) to 130-140 nm (Lac-S48I48) has been completed. Unique thrombin cleavage site design provides additional release route besides possible cleavage by protease existing in human tears. The thrombin cleavage site may comprise the amino acid sequence: GLVPRGSG (SEQ ID. NO: 7).

### ELP Protein Expression and Purification

Polynucleotides encoding ELPs S48I48 (SEQ ID NO: 1), S96 (SEQ. ID. NO: 2) and V96 (SEQ. ID. NO: 3) were expressed in BLR (DE3) E. coli cells (Novagen Inc., Milwaukee, Wis.). Briefly, after overnight start culture, protein was expressed for 24 h in an orbital shaker at 37° C. at 250 rpm. Cell culture were harvested and resuspended in phosphate buffer saline (PBS). After sonication and removing insoluble cell debris and nucleic, ELPs were purified from clarified cell supernatant by inverse transition cycling (ITC)15 as follows: the ionic strength of warmed-up soluble lysate (37° C.) was increased by adding crystal NaCl to trigger aggregation of ELPs. Aggregated protein was separated from soluble E. coli proteins by centrifugation at moderate temperature (37-40° C.). The pellet containing target protein was then dissolved in cold PBS on ice and centrifuged at 4° C. to remove any insoluble contaminants. This aggregation and dissolution process was repeated 6-7 times until proteins was determined to be approximately 99% pure by SDS-PAGE gels stained with coomassie blue. Protein concentrations were determined by UV-visible spectroscopy at 280 nm (ε=1285M−1 cm−1). Protein molecular weight is further confirmed by MALDI-TOF analysis.

### Fluorescein Labeling ELPs and Lacritin-ELPs

ELPs S48I48, S96 and V96 were conjugated with NHS-Rhodamine (Thermo Fisher Scientific Inc, Rockford, Ill.) via covalent modification of primary amines at the amino end of the peptide. Briefly, the conjugation was performed in 100 mM borate buffer (pH8.5) for overnight at 4° C., and conjugated protein was desalted using PD10 column (GE Healthcare, Piscataway, N.J.) and overnight dialysis against PBS at 4° C. For Lacritin and Lacritin-ELPs, the conjugation time was shortened to 2 h at 4° C. due to multiple Lysine residues in lacritin sequence.

Labeling Proclear Contact Lenses with Rho-ELPs

Proclear compatible contact lens was incubated in 100 μM-500 μM Rho-ELPs (Rho-V96, Rho-S548I48 or Rho-S96) for 48 hours at 4° C. or 37° C. After gentle rinse with PBS, contact lens was imaged using Zeiss 510 confocal microscopy or Bio-Rad VersaDoc MP System.

### Example 2

### Rhodamine Label ELPs and Contact Lens Decoration

Briefly, ELPs were covalently modified with NHS-Rhodamine (Thermo Fisher Scientific Inc, Rockford, Ill.) via the primary amino terminus. The conjugation was performed in 100 mM borate buffer (pH 8.5) overnight at 4° C. Excess fluorophore was removed using a desalting PD-10 column (GE Healthcare, Piscataway, N.J.) and overnight dialysis against PBS at 4° C. Contact lenses were either incubated with 50 μM labeled ELPs overnight at 37° C. in a 24-well plate or spot decorated with concentrated labeled ELPs using a 20 μl pipette at 37° C. before transferred to PBS solution.

### ELPs Inverse Phase Transition Characterization

The temperature-concentration phase diagrams for rhodamine labeled ELPs/ELP fusion proteins were characterized by optical density observation using a DU800 UV-Vis spectrophotometer at 350 nm as a function of solution temperature. Typically, ELPs (5-100 μM) were heated at 1° C./min from 10 to 85° C. and sampled every 0.3° C. Tₜ was defined at the point of the maximum first derivative.

### Fluorescence Release Characterization

ELP modified contact lens were gently rinsed with PBS and placed in 4 ml of PBS at 37° C. or 4° C. for 1 week. Samples of the solution (100 μl) were withdrawn at regular intervals and kept at −20° C. After one week, lenses were thoroughly washed in PBS at 4° C. for 24 hours to detach ELPs. Rhodamine intensity of collected samples was measured spectrophotometrically (Ex: 525 nm, Em: 575 nm) using Synergy™ Hlm Monochromator-Based Multi-Mode Microplate Reader (BioTek) and analyzed using Gen5 2.01 Data Analysis Software (BioTek). Total fluorescence on the lens was calculated using Equation 1. Retention rate was calculated using Equation 2. Raw data were then fitted into either a one phase decay model (Equation 3) or two phase decay mode (Equation 4) using SPSS. Goodness of fit and predicted values were collected.

$$\begin{matrix}Total {I}_{rhodamine} = {I}_{{release}_{Total}} + {I}_{{wash}_{Total}} & ( 1 ) \\ Retention ( \% ) = \frac{Total {I}_{rhodamine} - {E}_{t = 0}^{t} {I}_{{release}_{t}}}{Total {I}_{rhodamine}} \times 100 \% & ( 2 ) \\ Retention ( \% ) = ( {R}_{0} - Plateau ) \times {e}^{- kt} + Plateau & ( 3 ) \\ Retention ( \% ) = Plateau + {Span}_{fast} \times {e}^{- {k}_{fast} t} + {Span}_{slow} \times {e}^{- {k}_{slow} t} & ( 4 )\end{matrix}$$

### Spatiotemporal HCE-T Cell Uptake Study

HCE-T cell uptake study was conducted on 35 mm glass coverslip-bottomed dishes. Briefly, HCE-T cells were grown to 70-80% confluency and gently rinsed with warm fresh medium before changed to fresh medium containing either rhodamine labeled lacritin, Lac-V96 or Proclear Compatible™ contact lens modified with rhodamine labeled Lac-V96. After incubation at 37° C. for 1 hour, the cells were rinsed with fresh medium and images were immediately acquired using ZEISS 510 confocal microscope system. For uptake quantification comparison, images were analyzed using ImageJ.

### Statistical Analysis

Data presented are representative curves or mean±S.D. All experiments were repeated at least three times. Statistical analysis was performed by Student t-test or one-way ANOVA by SPSS. Differences between treatments were established with Tukey's post-hoc test. A p value of less than 0.05 was considered statistically significant.

### Discussion and Results

In an extension of Experiment No. 1, Applicant report the surprising discovery of ELPs' thermally-reversible, spatiotemporal and sustained attachment to Proclear Compatibles™ contact lens as an elastic bridge. Moreover, attachment and release of ELPs to/from Proclear contact lens was a Tₜ and temperature dependent process using rhodamine as a detection probe. For this study, two ELPs, V96 (Tₜ: ˜30° C.) and S96 (Tₜ: ˜55° C.) were used. When attaching the lens with V96 at 37° C., around 80% of fluorecence remained on the lens after one week incubation in PBS solution at 37° C., while the plateau of fluorescence retention dropped down to below 10% when releasing at 4° C. Lenses modified with S96 did not exhibit significant total fluorescence or release profile differences at either 37° C. or 4° C. Interestingly, lenses modified with V96 at 4° C. exhibited similar release pattern at 4° C. compared to S96 group, both of which can be described using a single two-phase decay model. The lens were further modified with prosecretory mitogenic fusion protein (Lac-V96) and demonstrated spatial cell uptake via contact lens using human corneal epithelial cell model (HCE-Ts).

### Discovery of ELPs Specific Attachment to Proclear Compatible™ Contact Lens

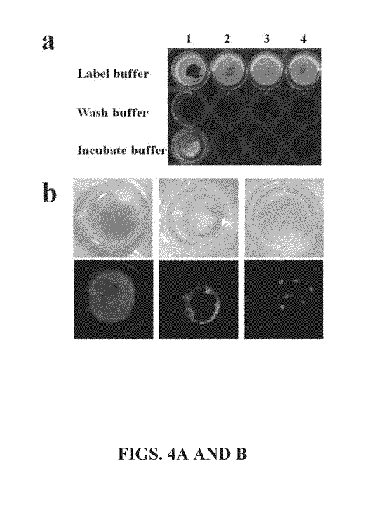

Surprising discovery of ELPs' attachment to Proclear Compatible™ contact lens came from a quick screen of four types of market contact lenses, including Acuvue Oasys®, Acuvue Advance Plus®, Dailies AquaComfort Plus™ and Proclear Compatibles™. Unexpectedly, rhodamine labeled V96 selectively attached to Proclear Compatibles™ contact lens at 37° C. after overnight incubation in PBS solution and the attachment was stable at 37° C. in PBS solution for more than 24 hours. Motivated by the rationale that the delivery system itself should not interfere with normal vision, Applicants investigated whether it was possible to spatially decorate the lens with ELPs. Interestingly, Applicants were able to modify the lens with various shapes of ELPs according to the need, such as ring, dots, etc (FIG. 4B).

### Tᵣ and Temperature Dependent Attachment of ELPs to the Lens

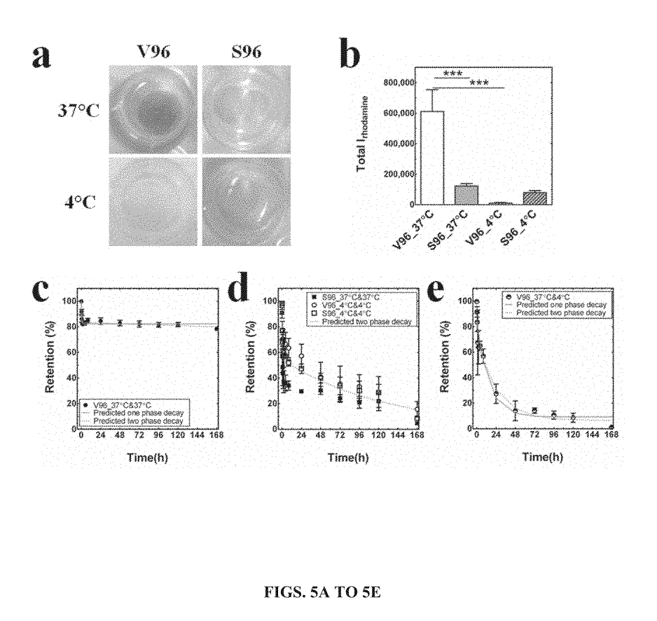

Without being bound by theory, Applicants proposed that the attachment of the ELPs to the contact lens was partially Tₜ and temperature dependent. To further explore the Tₜ and temperature dependence of ELPs' affinity to Proclear Compatible™ contact lens, Applicants chose two types of representative ELPs: V96 (Ti at around 30° C.) and S96 (Tₜ at around 55° C.) for a five group comparison study: i) Group one: lens incubated with V96 at 37° C. and release at 37° C. (closed circle); ii) Group two: lenses incubated with S96 at 37° C. and release at 37° C. (closed square); iii) Group three: lenses incubated with V96 at 4° C. and release at 4° C. (open circle); iv) Group four: lenses incubated with S96 at 4° C. and release at 4° C. (open square); v) Group five: lenses incubated with V96 at 37° C. and release at 4° C. (half closed circle). After 24 h incubation, total attachment of V96 at 37° C. (Group one) was about six fold of S96's attachment at 37° C. (Group two) and sixty-nine fold of V96's attachment at 4° C. (Group three) (FIGS. 5A and 5B). Interestingly, S96 incubated at 37° C. (Group two), V96 incubated at 4° C. (Group three) and S96 incubated at 4° C. (Group four) did not exhibit significant different contact lens attachment affinity (p>0.50) (FIGS. 5A and B). After one week release in PBS, only Group one exhibited around 80% of fluorescence retention on the lens while all the other groups released most of the attached ELPs (FIGS. 5C to 5E). Total fluorescence intensity provided the first clue of the association between contact lens affinity and Ti. To thoroughly compare fluorescence release kinetics of all five groups, Applicants fitted the data using both one phase decay and two phase decay models by SPSS (Table 2). Both Group one and Group five data can be described using a one phase decay model, with R² of 0.916 and 0.953 accordingly; while the other three groups did not fit the one phase decay model very well (R²=0.646). Interestingly, release kinetics of Group two, Group three and Group four can be described using the same two phase decay model (R²=0.847). The modeling result highly supported our hypothesis about the link between ELPs' attachment to Proclear Compatible™ contact lens and Tₜ/temperature. Most significant different release profile comes from Group one, which exhibited a predicted plateau of more than 75% retention after one week's incubation at 37° C. Retention of V96 (Group five) on the lens was significant lowered when the incubation temperature was changed to 4° C., with a predicted plateau of less than 10% using either model and a longer half-life of release (Table 2). The link between lens affinity and Tₜ was further corroborated by Group two, three and four. As when both incubation and release temperatures were below ELPs' Tₜ, no significant difference was noticed in either total binding fluorescence intensity (FIG. 5B) or release kinetic profiles (FIG. 5D).

TABLE 2
Modeling of release kinetics

| | Group 1 | Group 2 | Group 3 | Group 4 | Group 5 |
|---|---|---|---|---|---|
| ELP Type | V96 | S96 | V96 | S96 | V96 |
| Label Temp (° C.) | 37 | 37 | 4 | 4 | 37 |
| Release Temp (° C.) | 37 | 37 | 4 | 4 | 4 |

| Model (one | R = (R₀ − Plateau) * exp(−k * t) + Plateau | | |
|---|---|---|---|
| phase decay) | | | |
| Predicted | 100.048 ± 1.722 | 74.323 ± 4.784 | 85.585 ± 4.103 |
| R₀ (%) | | | |
| Predicted | 82.222 ± 0.576 | 29.728 ± 3.397 | 9.281 ± 3.956 |
| Plateau (%) | | | |
| k (h⁻¹) | 2.875 ± 0.653 | 0.174 ± 0.072 | 0.069 ± 0.018 |
| t_{1/2} (h) | 0.241 | 3.984 | 10.046 |
| R² | 0.916 | 0.646 | 0.953 |
| Model (two | R = Plateau + Span_{fast} * exp(−k_{fast} * t) + Span_{slow} * | | |
| phase decay) | exp(−k_{slow} * t) | | |
| Predicted | 75.000 ± 36.259 | 0.000 ± 27.790 | 6.436 ± 2.669 |
| Plateau (%) | | | |
| Predicted | 8.526 ± 35.885 | 40.503 ± 6.210 | 64.419 ± 4.209 |
| Span_{fast} (%) | | | |
| k_{fast} (h⁻¹) | 0.003 ± 0.017 | 3.301 ± 1.181 | 0.040 ± 0.008 |
| t_{1/2fast} (h) | 231.049 | 0.210 | 17.329 |
| Predicted | 16.563 ± 1.569 | 54.234 ± 26.585 | 30.398 ± 5.478 |
| Span_{slow} (%) | | | |
| k_{slow} (h⁻¹) | 3.362 ± 0.744 | 0.008 ± 0.007 | 1.943 ± 0.797 |
| t_{1/2slow} (h) | 0.206 | 86.643 | 0.357 |
| R² | 0.957 | 0.847 | 0.990 |

### Lac-V96 Ring Decorated Contact Lens Mediated Spatiotemporal HCE-T Cell Uptake

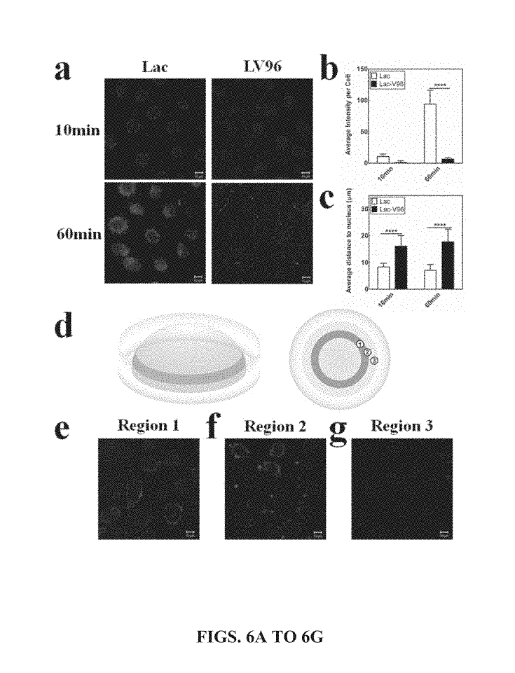

To explore the targeted delivery potential of ELP-contact lens system, Applicants chose one of the potential protein therapies for ocular disease, lacritin, which has shown prosecretory mitogenic activities as dry eye disease and cornea wound healing treatment. Applicants have previously proved that Lac-ELP fusion proteins imparted similar proseceretroy/mitogenic function of lacritin and thermo responsiveness of ELPs. Moreover, by fused to different ELP tags, uptake level and speed of exogenous Lac-ELPs into HCE-Ts could be modulated (FIGS. 6A-C). The spatiotemporal controlled HCE-T cell uptake effect was enhanced when the lens was ring decorated with rhodamine labeled Lac-V96 (FIG. 6D). Three representative regions underneath the lens were chosen to compare cell uptake level and distribution (FIGS. 6E-G). As illustrated in the figures, Lac-V96 ring decorated contact lens successfully executed its targeted delivery task. Region 1 (FIG. 6E) was fully covered by the lens, exhibiting evenly distributed highest cell uptake level. Region 2 (FIG. 6F) was partially covered by the lens and thus only showed one section of cell uptake. Region 3 (FIG. 6G) was outside of the Lac-V96 ring area, which illustrated the lowest cell uptake level.

To develop new treatments or delivery mechanisms for ocular diseases and improve the bioavailability, new drug vehicles are required to be biocompatible, biodegradable, easily modified with bioactive peptides, small molecules or antibodies and can work in concert with existing medical devices to provide novel functionality. In this communication, Applicants reported the surprising discovery of thermal responsive ELPs' selective reversible attachment to Proclear Compatibles™ contact lens; Applicants described the Tₜ and temperature dependence of this attachment and Applicants provided the first proof of concept to spatiotemporally deliver model ocular protein drug lacritin via contact lens. Different from reported contact lens mediated drug delivery systems, the ELP modification on contact lens can be Tₜ and spatiotemporally modulated so that delivery is more targeted to the disease site and delivery rate can be further fine-tuned using external stimuli such as local cooling for on demand dosing. In this study, the monoblock ELP modified contact lens was fused with fluorescent labeled therapeutic agent for visual detection of release and in vitro cell uptake.

It should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

1. A biocompatible, polymeric material comprising an elastin-like peptide (ELP).

2. The polymeric material of claim 1, wherein the ELP is attached to the polymeric material randomly or in a pre-determined design.

3. The polymeric material of claim 1 or 2, wherein the ELP comprises one or more of SEQ ID NOs: 1 to 6, or a biological equivalent thereof.

4. The polymeric material of claim 1 or 2, wherein the ELP further comprises a therapeutic agent bound to the ELP or encapsulated within the ELP.

5. The polymeric material of claim 4, wherein the therapeutic agent is lacritin or a biological equivalent thereof.

6. The polymeric material of claim 4, wherein lacritin is fused to the ELP.

7. The polymeric material of claim 4, further comprising a cleavable peptide sequence located between the therapeutic agent and the ELP.

8. The polymeric material of claim 6, further comprising a cleavable peptide sequence located between the therapeutic agent and the ELP.

9. The polymeric material of claim 7, wherein the cleavable peptide sequence is a thrombin cleavable peptide sequence.

10. The polymeric material of claim 8, wherein the cleavable peptide sequence is a thrombin cleavable peptide sequence.

11. The polymeric material of claim 5, wherein the lacritin protein comprises an amino acid sequence corresponding to the amino acid sequence of SEQ ID NO: 8 or 10 or a biological equivalent thereof.

12. The polymeric material of claim 11, wherein the lacritin-ELP comprises an amino acid sequence corresponding to the amino acid sequence of SEQ ID NO: 9 or a biological equivalent thereof.

13. The polymeric material of any one of claims 1-9, further comprising a therapeutic agent.

14. The polymeric material of claim 4, wherein the therapeutic agent is an anti-microbial agent or a non-steroidal anti-inflammatory drug.

15. The polymeric material of claim 1, further comprising a detectable label.

16. The polymeric material of claim 1, wherein the ELP comprises a diblock.

17. A method for preparing the polymeric material of claim 1 comprising absorbing, conjugating, or coating a polymeric material with an ELP.

18. A method for delivering a therapeutic agent, comprising contacting the polymeric material of claim 4 with a subject to be treated.

19. The method of claim 18, wherein the polymeric material is in contact with the ocular surface of an eye.

20. A method for treating an ocular disease, comprising contacting the polymeric material of claim 4 with the eye of a patient in need of such treatment.

21. The method of claim 20, wherein the ocular disease is selected from the group consisting of dry eye, age-related macular degeneration, diabetic retinopathy, retinal venous occlusions, retinal arterial occlusion, macular edema, postoperative inflammation, infection, dryness, uveitis retinitis, proliferative vitreoretinopathy and glaucoma.

22. The method of claim 21, wherein the ocular disease is dry eye.

23. A polypeptide comprising the amino acid sequence corresponding to the amino acid sequence of SEQ ID NO: 9 or a biological equivalent thereof.

24. A method for delivering a therapeutic agent, comprising contacting the polymeric material of claim 23 with a subject to be treated.

25. The method of claim 24, wherein the polymeric material is in contact with the ocular surface of an eye.

26. A method for treating an ocular disease, comprising contacting the polymeric material of claim 23, with the eye of a patient in need of such treatment.

27. The method of claim 26, wherein the ocular disease is dry eye.

28. A polynucleotide encoding the polypeptide of claim 23.

29. A host cell comprising the polynucleotide of claim 24.

30. A composition comprising a carrier and the polypeptide of claim 23.

31. A method for preparing the polypeptide of claim 23, comprising expressing the polynucleotide of claim 24.

32. A method for preparing the polypeptide of claim 23, comprising expressing the polynucleotide of claim 24 in the host cell of claim 25.

33. The method of claim 32, further comprising separating or purifying the drug delivery agent.

34. A kit comprising one or more polymeric materials of any one of claims 1 to 16 and instructions for use.
